(19) United States
(12) Patent Application Publication
(10) Pub. No.: US 2005/0043690 A1
(43) Pub. Date: Feb. 24, 2005

(54) Cannula that provides bi-directional fluid flow that is regulated by a single valve

(75) Inventor: Erik F. Todd, Brisbane, CA (US)

Correspondence Address: FLYNN, THIEL, BOUTELL & TANIS, P.C., 2026 RAMBLING ROAD, KALAMAZOO, MI, 49008-1631, US

(73) Assignee: Stryker Corporation

(21) Appl. No.: 10/949,957

(22) Filed: Sep. 24, 2004

Related U.S. Application Data
(63) Continuation of application No. 09/952,669, filed on Sep. 12, 2001, now abandoned.

(51) Int. Cl. A61M 5/00

(52) U.S. Cl. 604/248

(57) ABSTRACT

A cannula useful for endoscopic surgery. The cannula includes an outer tube in which an inner tube is removably seated. A surgical instrument can be positioned at the surgical site through the inner tube and irrigation fluid can be flowed to the surgical site through the inner tube. The tubes are spaced from each other to form a channel through which fluid can be selectively withdrawn from the surgical site. Fluid flow to and from the surgical site through the cannula is controlled by a valve assembly that has a single valve cock. The valve cock can be positioned to allow maximum irrigation flow/no suction flow or no irrigation flow/maximum suction flow. Bores internal to the valve cock are shaped so that, while the suction flow is initially incrementally increased, the irrigation flow is at a constant level.

This application is a continuation of U.S. Ser. No. 09/952,669, filed Sep. 12, 2001.

### FIELD OF THE INVENTION

This invention is related generally to a cannula useful for arthroscopic surgery. More particularly, this invention is directed to a cannula through which fluid can simultaneously be infused to and withdrawn from the surgical site and that has a single valve control that regulates fluid flow in both directions.

### BACKGROUND OF THE INVENTION

Over the last decade it has become popular to perform surgical procedures endoscopically. In an endoscopic surgical procedure, a device known as an endoscope, which is in the form of an elongated tube, is placed in a body cavity or into a joint and positioned at the site where the surgical procedure is to be performed. The endoscope allows the surgeon to view the surgical site on a video monitor. Other surgical instruments are inserted into the body cavity or the joint for manipulation or removal of tissue. The surgeon views the surgical site via the endoscope while manipulating the other instruments to perform the desired surgical procedure. The development of endoscopes and their associated surgical instruments has made it possible to perform minimally invasive surgery. This type of surgery eliminates the need to make a large incision to gain access to the surgical site. Instead of having to make large incisions, endoscopic surgery entails making small openings, called portals. The endoscope and other surgical instruments are inserted through these portals. An advantage of performing endoscopic surgery is that this technique minimizes tissue trauma, which both greatly hastens postoperative healing time and greatly reduces postoperative pain. Additionally, endoscopic surgery is advantageous in that it exposes very little of the patient's tissue to the operating room environment. This greatly reduces susceptibility of the tissue to infection.

An important subcategory of endoscopic surgery is known as arthroscopic surgery. By definition, arthroscopic surgery is endoscopic surgery that is performed on a joint, such as the knee, shoulder, or elbow. Arthroscopic surgery has been technically enhanced by the development of fluid management systems. A fluid management system pumps a clear, sterile fluid solution into the joint at which the surgery is performed. Since the joint is a relatively tightly enclosed space, the fluid remains contained within that joint. The fluid surrounds and expands the space within the joint and the adjacent soft tissues so as to increase both the field of view of the surgical site and the space available for the manipulation of the surgical instruments. The fluid also serves to control and flush away blood and other debris that may obscure the view of the joint.

Currently, fluid management systems include two tube-shaped cannulae. A first cannula is placed into the joint and functions as the conduit through which the fluid management system applies fluid into the surgical site. The second cannula, fitted in a separate portal formed in the body, serves as the conduit through which the fluid is drawn from the surgical site. To minimize the number of portals that are formed in the patient's body, the first inflow cannula also typically functions as the member through which the endoscope is inserted. Thus, this cannula directs the endoscope to the desired field of view. A disadvantage of this arrangement is that it requires two separate portals to be formed, one for each cannula.

Moreover, the surgeon seldom maintains the fluid flows into and out of the two cannulae at constant rates. Throughout much of the procedure, a considerable amount of the fluid introduced into the joint space is drawn out of the site via a suction bore in the instrument the surgeon applies to the site. This arrangement enables the surgeon to regulate the pressure within the joint at a desired level. In this “steady-state” operation of the fluid management system, very little fluid may be drawn out via the outflow cannula. However, during the course of a procedure, blood, other fluid and debris can and often do obscure the visualization of the joint. In order for the surgeon to obtain a clear view of the surgical site, it is necessary for the surgeon to flush out the fluid and debris. This process is performed by allowing fluid to flow out through the outflow cannula and/or momentarily reducing or shutting off fluid flow into the joint via the inflow cannula. To accomplish these tasks, the surgeon must manipulate separate valves attached to the individual cannulae.

A disadvantage of the above process of fluid control is that it is ergonomically awkward. Also, the surgeon must devote attention to the appropriate, essentially simultaneous setting of two valves to perform the desired flushing of the surgical site. This causes the surgeon to have to cease performing other steps of the surgical procedure and direct attention away from the video monitor. The surgeon then is required to regain concentration in order to return the interrupted tasks of the procedure. Thus, the current process requires that the surgeon multitask between the field of interest and the motor control of the existing instrumentation. This, in turn, prolongs the operating time to complete the intended surgical procedure. In modern medicine, it is desirable to perform a surgical procedure so as to both utilize resources and personnel expeditiously as possible and to minimize the amount of time the patient is held under anesthesia.

### SUMMARY OF THE INVENTION

This invention relates to a cannula into which an endoscope is inserted, and which has separate conduits through which fluid can be simultaneously introduced into and withdrawn from the surgical site (for example, a joint). The cannula of this invention also has a single valve, with a single control member that variably regulates both the inflow of fluid into the surgical site through the cannula and the fluid withdrawn from the site through the cannula.

### BRIEF DESCRIPTION OF THE DRAWINGS

This invention is pointed out with particularity in the claims. The above and further features of this invention may be better understood by reference to the following description taken in conjunction with the accompanying drawings in which:

FIG. 1 depicts how the cannula of this invention is used to facilitate the performance of an endoscopic surgical procedure;

FIG. 2 is a perspective view of the cannula of this invention;

FIG. 3 is an exploded view of the cannula;

FIG. 4A is a perspective view of the outflow body of the cannula;

FIG. 4B is a cross sectional view of the outflow body taken along lines 4B-4B of FIG. 4A;

FIG. 5 is a perspective view of the outflow cap of the cannula;

FIG. 6 is a perspective view of the outflow slide;

FIG. 7 is a cross sectional view of the inner tube of the cannula;

FIG. 8A is a plan view of the inflow housing;

FIG. 8B is a cross section of the inflow housing taken along lines 8B-8B of FIG. 8A;

FIG. 9A is a perspective view of the inflow plug;

FIG. 9B is a cross sectional view of the inflow plug taken along line 9B-9B of FIG. 9A;

FIG. 10 is a perspective view depicting the arrangement of the inner tube, the inflow housing and the inflow plug;

FIG. 11A is a perspective view of the collet;

FIG. 11B is a cross-sectional view of the collet taken along line 11B-11B of FIG. 11A;

FIG. 12 is a cross sectional view of the proximal end of the cannula of this invention;

FIG. 13 is an exploded view of the valve assembly of the cannula;

FIG. 14 is a cross sectional view of the valve body taken along line 14-14 of FIG. 12;

FIG. 15 is a plan view of the valve cock; and

FIG. 16 is an end view of the distal end of the cannula.

### DETAILED DESCRIPTION

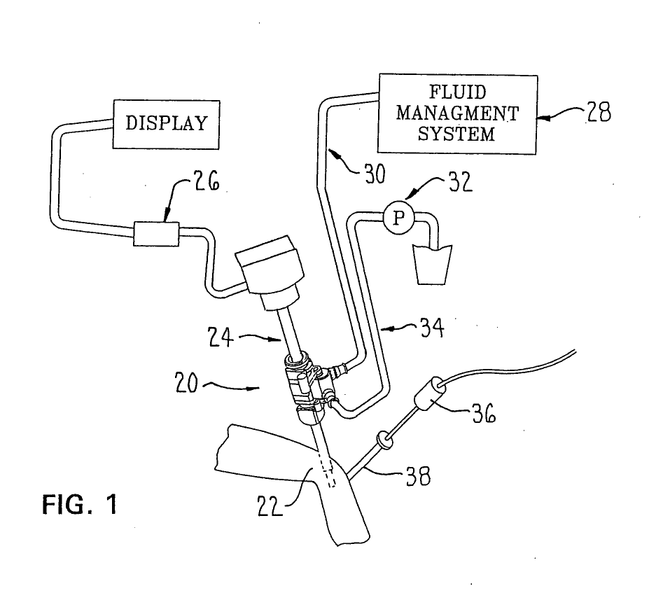

FIG. 1 depicts how a cannula 20 of this invention is used to facilitate the performance of an endoscopic surgical procedure. The cannula 20 is positioned in the patient's body at a location at which the surgical procedure is to be performed, in FIG. 1, a knee 22. An endoscope 24 is fitted in the center of the cannula 20. A surgeon views the surgical site on a camera-display system 26 fitted to the endoscope 24. A fluid management system 28 is also connected to the cannula 20. The fluid management system 28 includes a pump, not illustrated, that forces sterile solution through an inflow line 30 into the cannula 20. The fluid management system 28 includes a suction pump 32 that is connected to the cannula through an outflow line 34. The fluid introduced into the surgical site through the inflow line 30 and cannula 20 is selectively drawn away from the site through the cannula 20 and the outflow line 34 by suction pump 32. Also seen in FIG. 1 is a powered handpiece 36 that is positioned at the surgical site through a supplemental cannula 38. The powered handpiece 36, not part of this invention, includes a cutting accessory, not identified, that is applied to the surgical site in order to perform the intended surgical procedure. A suction path, formed by conduits internal to the cutting accessory and the handpiece 36, is connected to the suction pump 32. Often, during a surgical procedure, suction pump 32 continually draws fluid from around the distal end of the cutting accessory, the end position at the surgical site, out through the accessory and the handpiece 36.

(For point of reference in this application, “distal” shall refer to the direction towards the surgical site. “Proximal” shall refer to the direction away from the surgical site, towards the surgeon performing the surgical procedure.)

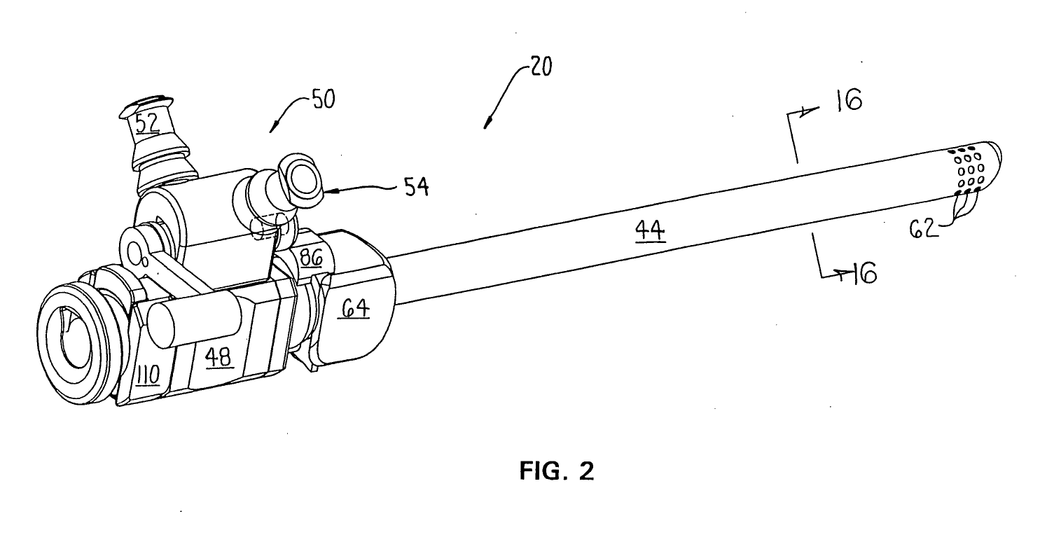

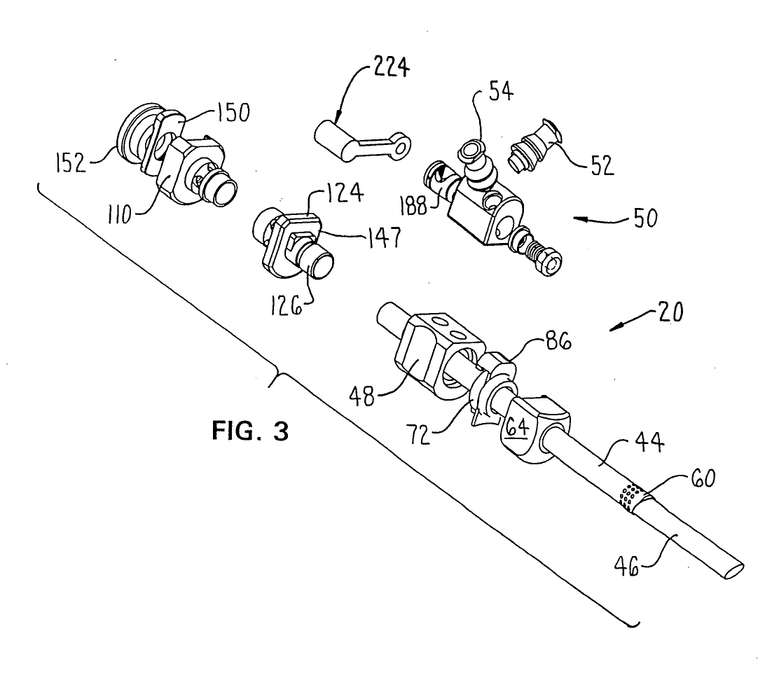

As seen in FIGS. 2 and 3, cannula 20 includes an outer tube 44 and an inner tube 46 disposed in the outer tube 44. Outer and inner tubes 44 and 46 form the distal end of the cannula 20, the end of the cannula that is fitted in the body of the patient. The distal end of the endoscope 24 is seated in the inner tube 46 so that the endoscope can be properly positioned at the surgical site. The inner tube 46 is dimensioned so that there is an annular gap between its inner wall and the outer wall of the complementary endoscope 24. Fluid is introduced into the surgical site through this gap. Collectively, the outer and inner tubes 44 and 46, respectively, are dimensioned so that there are one or more curved gaps between the tubes. These gaps, as discussed below, function as channels through which fluid is selectively drawn away from the surgical site.

The inner tube 46 extends outward beyond the proximal end of the outer tube 44. A collet 48 is mounted around a section of the exposed portion of the inner tube 46. A valve assembly 50 is attached to the collet 48. A first luer fitting 52, attached to valve assembly 50, serves as the connector between the fluid inflow line 30 and the cannula 20. A second luer fitting 54, also attached to valve assembly 50, functions as the connector between fluid outflow line 34 and the cannula 20. Valve assembly 50 has a single, rotatable valve cock 188. Valve cock 188 is manually set to both establish the inflow fluid flow rate through the cannula 20 to the surgical site and the outflow flow rate from the surgical site through the cannula 20.

Outer tube 44 has a circular cross-sectional profile through the length of the tube. It is anticipated that in many versions of the invention the outer diameter of the outer tube will be between 2 and 15 mm and the tube will have a wall thickness of 0.004 to 0.015 inches. The distal end tip of the outer tube 44 is shaped to have flare 60. Flare 60 is provided to facilitate the insertion of the outer tube 44 in the patient. Proximal to the distal end of the outer tube 44, the tube 44 is provided with a ring of apertures 62. Apertures 62 serve as ports through which fluid that is to be removed from the surgical site is drawn into the cannula 20.

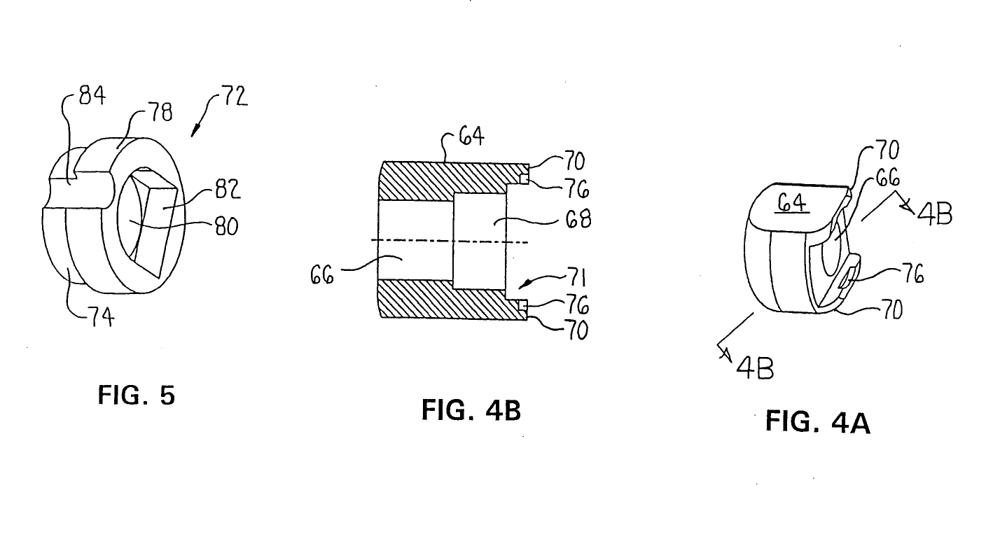

The proximal end of the outer tube 44 is permanently secured to an outflow body 64 best illustrated by FIGS. 4A and 4B. The outflow body 64 is a solid piece of metal. The outflow body 64 is formed to have a bore 66 that extends proximally from the distal facing face of the body. Bore 66 opens into a larger counterbore 68. Counterbore 68 opens into the proximal facing face of the outflow body 64. The proximal end of the outer tube 44 is welded or otherwise permanently secured to the inner wall of outflow body 64 that forms bore 66.

Outflow body 64 is further formed to have two linearly shaped lips 70 that extend away from the proximal face of the body. Lips 70 are located on opposed sides of the counterbore 68. Lips 70 thus define an elongated slot 71 in front of the opening into counterbore 68.

An outflow cap 72, shown in detail in FIG. 5, is welded or otherwise permanently secured to lips 70 of outflow body 66. The outflow cap 72 is generally circular in structure. The outflow cap has a base 74. It will be observed that outflow body lips 70 are formed with opposed arcuately shaped cutouts 76. The distal end of outflow cap base 74 is seated in the lip cutouts 76. Thus, outflow cap 72 is spaced away from the proximal-end face of outflow body 64.

Outflow cap 72 is also shaped to have a head 78 that is integrally formed with and has a larger outer diameter than the base 74. The outflow cap 72 is formed with a bore 80 that extends axially through the cap. Cap head 78 is further formed to define a three-sided void space 82 that is contiguous with bore 80. Outflow cap is further formed so that the outer surface of the base 78 and head define a single, longitudinally extending, arcuately shaped slot 84.

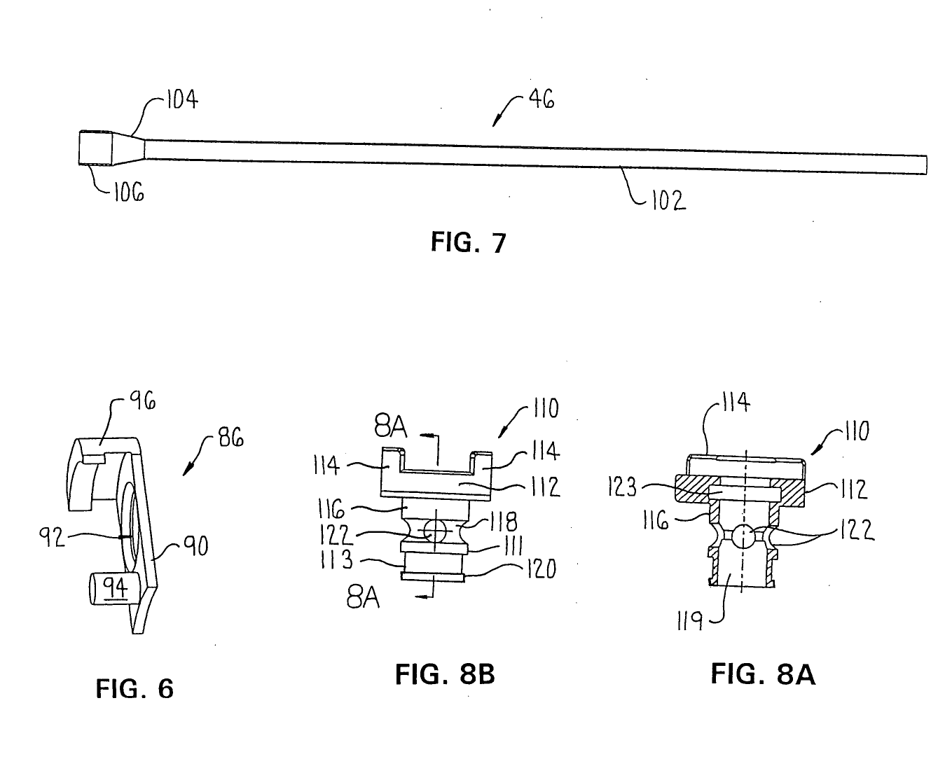

An outflow slide 86, illustrated in FIG. 6, is slidably fitted in slot 71 between outflow body 64 and outflow cap 72. Outflow slide 86 has a flat plate 90 that is slidably fitted against the proximal face of outflow body between lips 70. The plate 90 is formed with an opening 92. A pin 94 extends rearwardly from the proximal face of the plate 90 at one end of the body. A tab 96 is fitted over the opposed end of the plate 90. Tab 96 extends perpendicularly relative to the plate and over the outflow cap 72.

When the cannula 20 is assembled, the outflow slide 86 is positioned so that pin 94 seats in slot 84 formed in outflow cap 72. An O-ring 98 (FIG. 12) extends around the outside of cap base 74 and the exposed surface of pin 94. As described hereinafter, output slide 86 and O-ring 98 cooperate to releasably hold the inner tube 46 and the components with which it is integral to the outer tube 44.

The inner tube 46, while formed from a single piece of metal, has three distinct sections as seen in FIG. 7. A first section, the distal section 102, has an oblong or oval cross-sectional shape as seen in FIG. 16. Section 102 comprises the portion of the inner tube 46 that extends from the distal end of the tube, through the outer tube 44, the outflow body 64 and outflow cap 72 and a small distance forward of the outflow cap. The inner tube is dimensioned so that its major, widest outer diameter is equal to the inner diameter of the outer tube in which the inner tube is seated. The narrow, minor diameter of the inner tube is dimensioned to allow an endoscope or other instrument to be seated in the tube. It is anticipated that this minor diameter will be between 0.5 and 13.5 mm and the tube itself will have a wall thickness between 0.002 and 0.015 inches.

Inner tube 46 is further dimensioned so that when the cannula 20 is assembled, the distal end of the inner tube occupies the space subtended by apertures 62 in the outer tube 46. The opposed section of the inflow tube 46, the proximal section 106, has a circular cross sectional shape. Section 106 forms the proximal end of the inner tube 46. Between sections 102 and 106, the inner tube has a section 104. Section 104 tapers between the circular profile of section 106 and the non-circular profile of section 102.

Two methods of fabricating inner tube 46 are contemplated. In a first method, the raw workpiece, a circular tube, is ram fitted in a die. The die is shaped to deform the workpiece so that the section of the workpiece in the die is deformed to have the desired non-circular shape of section 102. Not all of the workpiece is fitted in the die. A portion is left outside so that the circular cross sectional profile of section 106 is maintained. A portion of the workpiece between sections 102 and 106 develops the tapered profile of section 104.

In the second method of manufacture, a circular tube is again employed as the stock workpiece. The end of the workpiece selected to become section 102 is placed in a vice. This section of the workpiece is compressed until it develops the desired non-circular profile.

A proximal portion of section 102, section 104 and section 106 of inner tube 44 extend out of the outflow cap 72. Section 106 is welded or otherwise secured inside an inflow housing 110, shown in detail in FIGS. 8A and 8B. The inflow housing 110, which is formed from a single piece of metal, has a flat head 112. Extending forwardly, from the proximal facing face of head 112, inflow housing 110 has two lips 114 located on opposed sides of the head 112. Extending distally from head 112, inflow housing 110 has a sleeve-like stem 116. The stem 116 is formed so that adjacent the head 112, there is a circumferentially extending groove 118. Extending distally from groove 118, stem 116 has a section 111 with a relatively large outer diameter followed by a section 113 with a reduced diameter. At the distal end, stem 116, is formed to have a lip 120 with an outer diameter between that of sections 111 and 113.

Inflow housing 110 is further formed to have a bore 119 that extends longitudinally through the housing, from head 112 to the stem distal end stepped section 120. Internal to the head 112 the inflow housing is formed to have an internal groove 123 that surrounds bore 119. A seal, not illustrated, is seated in groove 123. When the endoscope 24 is fitted in inner tube 46 and bore 119, the seal provides a liquid tight barrier around the portion of the endoscope seated in the inflow housing 110.

The inner tube proximal end section 106 is welded or otherwise permanently secured into the portion of inflow housing bore 119 defined by reduced diameter section 113 and lip 120. The inflow housing 110 is further formed to define four equiangularly spaced apart apertures 122 in groove 118. Apertures 122 provide fluid communication paths from outside of the inflow housing 110 into bore 119 and inner tube 44.

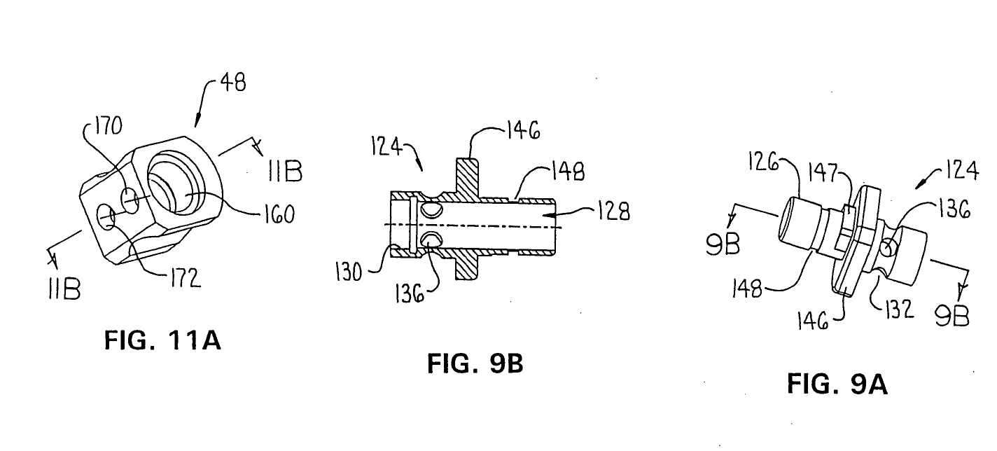

An inflow plug 124 is securely fitted to the inflow housing stem-stepped section 120 and extends over section 104 and the adjacent proximal end of section 102 of the inflow tube 46. The inflow plug 124, best seen in FIGS. 9A and 9B, has a generally sleeve-shaped body 126. A generally constant diameter bore 128 extends axially through body 126. The inflow plug 124 is further formed so as to have a counterbore 130 in the proximal end opening to bore 128. Stem reduced diameter section 113 is secured in and seats against the wall of counterbore 130. Extending distally along the plug 124, it will be observed that the outer surface of the body is formed to define a circumferentially extending groove 132. Four equiangularly spaced apart apertures 136 are formed in the body 126 and extend from groove 132 to bore 128.

The inflow plug 124 is further formed to have an end plate 146 that is formed integrally with the body 126 and lies in a plane perpendicular to the longitudinal axis of bore 128. The opposed faces of end plate 146 are flat. The distal-facing face of end plate 146 is, however, provided with a distally-directed three-sided nose 147 that partially surrounds the adjacent outer surface of plug body 126. Located distally from end plate 146, inflow plug body 126 is formed to have along its outer surface a circumferentially extending groove 148.

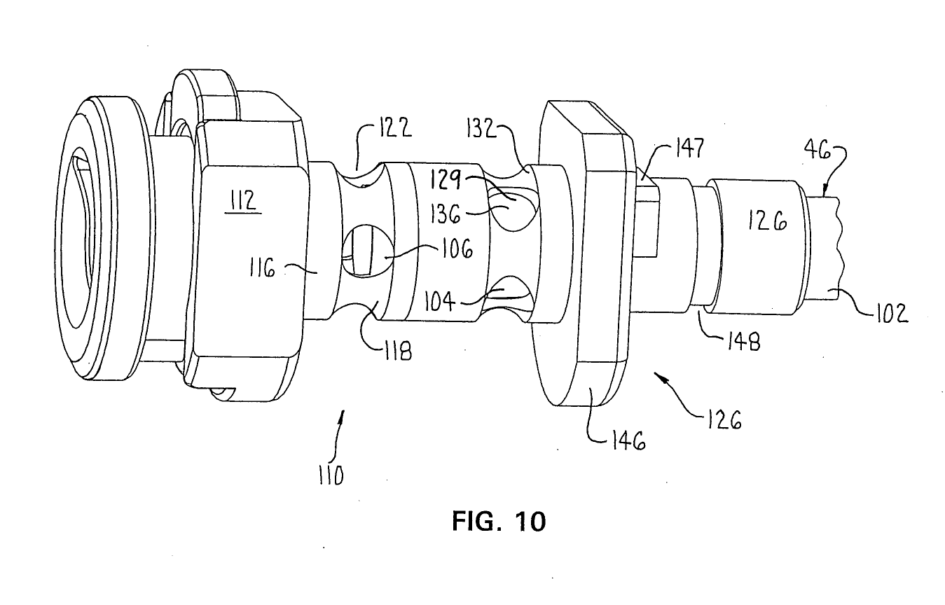

FIGS. 10 and 12 illustrate how inner tube 46, inflow housing 110 and inflow plug 124 are assembled together to form a head end of the cannula 20. The proximal section 106 of the inner tube 46 is welded or otherwise secured to the inner wall of the inflow housing 110 that defines the distal end of bore 120. The proximal end of the inflow plug body is seated over the inflow housing 110 so that sections 111 and 113 and lip 120 of the housing seat in plug counterbore 130. An annular seal 121 extends between the outer surface of housing section 113 and the adjacent inner wall of the plug 124 that defines the counterbore 120.

It will be further understood that inner tube 46 and inflow plug 124 are so shaped so that the plug bore 128 has a diameter greater than that of tube sections 102 and 104. Thus, there is an annular space 129 between the inflow plug 124 and the portions of tube sections 102 and 104 located within plug bore 128. When the inflow plug 124 is positioned over the inner tube 46, the portion of the plug that defines groove 132 subtends tube section 104. Apertures 136 thus serve as fluid communication paths from the space in plug bore 128 to the outside of the inflow plug 124.

A plate-shaped endoscope slide lock 150 is slidably located between lips 114 of the inflow housing 110. An outer cap or eyepiece 152 is secured to the inflow housing lips 114 and extends over the slide lock 150. An O-ring 154 extends around the eyepiece 152 and a pin integral with the slide lock 150 so as to normally hold the slide lock 150 in a static position relative to the rest of the inflow housing 150. Collectively, the inflow housing 110, the slide lock 150, the eyepiece 152 and the O-ring 154 form an assembly for releaseably holding the endoscope 24 in the cannula 20. A more detailed discussion of how this mechanism operates is found in the Applicant's Assignee's U.S. Pat. No. 5,456,673, LOCKING CANNULA FOR ENDOSCOPIC SURGERY, issued 10 Oct. 1995 and U.S. Pat. No. 5,810,770, FLUID MANAGEMENT PUMP SYSTEM FOR SURGICAL PROCEDURES, issued 22 Sep. 1998, both of which are incorporated herein by reference.

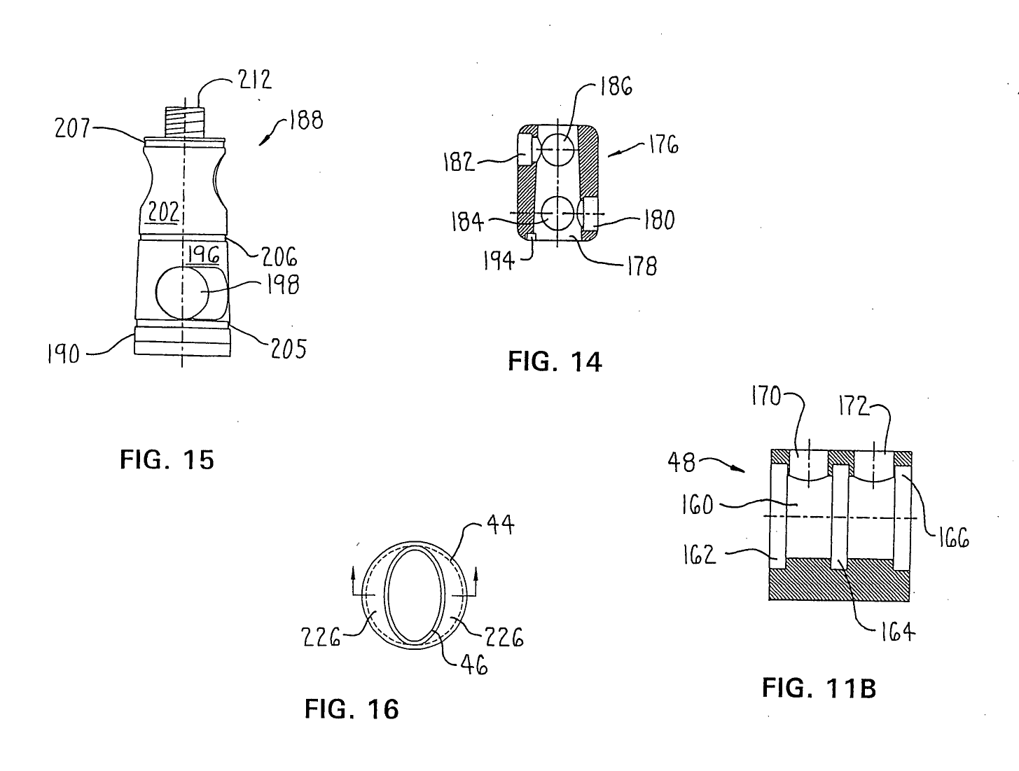

The collet 48, seen best in FIGS. 11A, 11B, and 12, is rotatably fitted over the inflow housing 110 and the inflow plug 124 between housing head 112 and plug end plate 146. Collet 48 is formed from a single piece of metal that is formed with a longitudinally extending through bore 160. The housing stem 116, the proximal portion of the plug body 126, and the sections of the inner tube 46 enclosed within these components are seated within the collet bore 160. The inside of the collet is provided with three, longitudinally spaced apart grooves 162, 164, and 166 that extend outwardly from bore 160. Groove 162 is located adjacent the proximal end of the bore 160. Groove 164 is located in the portion of the collet 48 that subtends the portion of the proximal end of the plug body 126 that overlaps inflow housing stem reduced diameter section 120. The remaining groove, groove 166 is located adjacent the distal end of bore 160. An O-ring or seal 168 is fitted in each groove 162, 164 and 166. The seals 168 in grooves 162 and 166 prevent fluid flow out of the ends of bore 160. Seal 168 in groove 164 prevents fluid leakage between the inflow and outflow fluid paths through the cannula 20.

Collectively, the components of the cannula 20 are dimensioned to allow the collet 48 and attached valve assembly 50 to rotate around the inflow housing and plug 110 and 124, respectively.

The collet is formed with circularly shaped inflow and outflow ports 170 and 172, respectively. The inflow port 170 is positioned to subtend inflow housing groove 118 and the associated apertures 122. The outflow port 172 is positioned to subtend inflow plug groove 132 and the associated apertures 136.

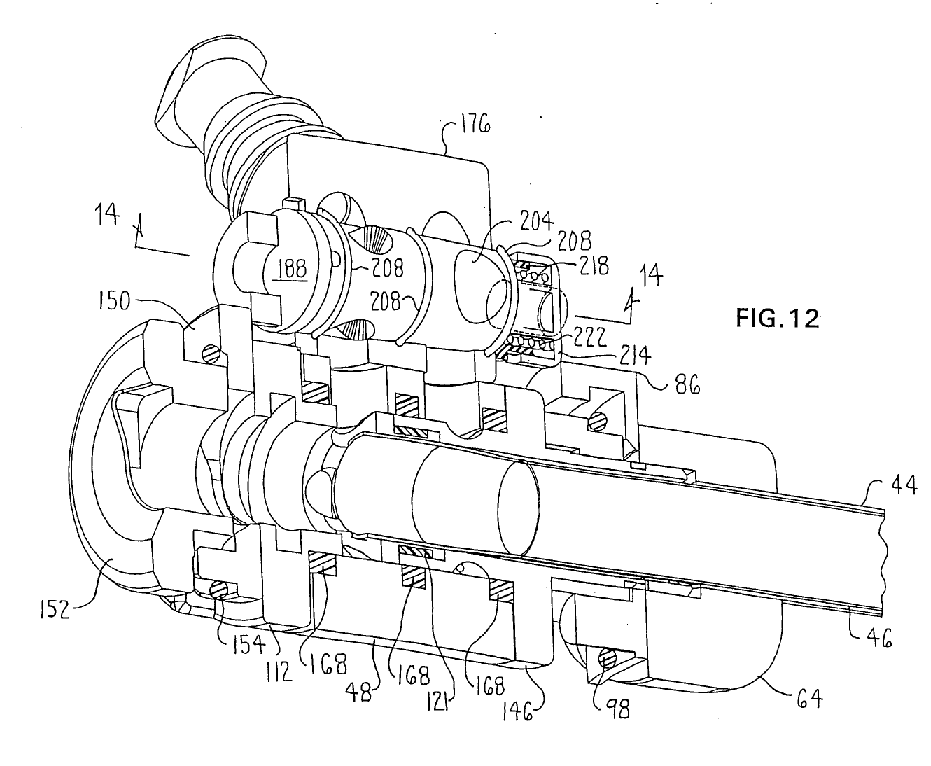

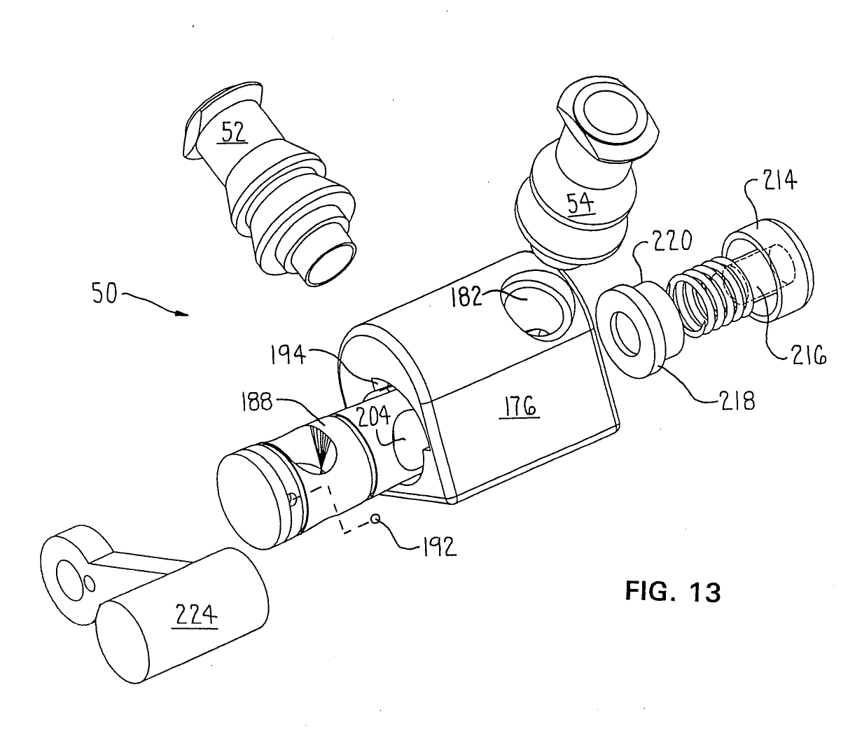

The valve assembly 50, best seen in FIG. 13, includes a valve body 176 that is fixedly secured to the collet 48. The valve body 176, seen in cross section in FIG. 14, is a solid piece of metal. The valve body 176 is formed with a longitudinally extending through bore 178. Bore 178 is tapered so as to have a wide diameter adjacent the proximal end of the valve body 176 and a narrower diameter adjacent the distal end. Adjacent its proximal end, an inlet hole 180 is provided on a first side of the valve body. Inlet hole 180 serves as the aperture in which luer fitting 52 is seated and placed in communication with bore 178. Adjacent its distal end an outlet hole 182 is provided on a second side of the valve body 176 opposite the first side. Outlet hole 182 functions as the aperture in which luer fitting 54 is seated and placed in communication with bore 178.

The bottom of the valve body 176 is formed to have spaced apart inflow and outflow ports 184 and 186, respectively. Inlet port 184 is laterally aligned with inlet hole 180. Outlet port 186 is laterally aligned with outlet hole 182. Valve body 176 is further shaped so that, when it is fitted against the collet 48, the valve body inlet port 184 is in registration over collet inlet port 170 and the valve body outflow port 186 is in registration over collet outflow port 172. Two open-ended tube-shaped sleeves, not illustrated, provide fluid communication between the collet 48 and the valve body. A first sleeve extends between collet and valve body inflow ports 170 and 184, respectively. A second sleeve extends between the collet and valve body outflow ports 172 and 186, respectively. Both sleeves are press fit into the ports into which they are seated so as to hold the valve body 176 to the collet 48.

A valve cock 188, formed from a single piece of metal, and best seen in FIG. 15, is rotatably seated in valve body bore 178. The valve cock 188 has a head end 190 that seats in the proximal end of valve body bore 178. A spring-loaded ball 192 is mounted in a laterally extending opening 193 formed in the valve cock head end 190. When the cannula 20 is assembled, ball 192 seats in an arcuate groove 194 formed in the proximal facing face of the valve body that is contiguous with bore 178. The degree of rotation of valve cock 188 is limited by the extent to which ball 192 is free to rotate within groove 194. In some preferred versions of the invention, the surface of valve body 176 that defines groove 194 is formed with ridges or other perturbations. These perturbations impose a resistance on the rotation of the ball 192 and, hence, the valve cock 188. This resistance functions as a tactile feedback that provides the surgeon of an indication of the position of the valve cock 188.

Extending distally from head end 190, the valve cock 188 is shaped to have an inlet section 196. Inlet section 196 has the same outer diameter as head end 190. The inlet section 196 is formed with a bore 198. Bore 198, is not cylindrically shaped. The opposed ends of the bore 198 are flared outwardly. Extending distally from the inlet section 196, the valve cock 188 is formed to have an outlet section 202. The outlet section 202 has an outer diameter that is tapered relative to the outer diameter of the head end 190 and inlet section 196. Outlet section 202 is formed to have a cylindrically shaped bore 204.

Three grooves 205, 206, 207 are formed in the valve cock 188. A first groove 206 is located immediately forward of bore 198. A second groove 206 is located between inlet and outlet sections 196 and 202, respectively. A third groove 207 is located distal to bore 204. O-rings 208 are seated in each groove 206 to serve as barriers that prevent leakage between the valve cock 188 and valve body 176.

A threaded stud 212 extends out from the distal-facing facing of the valve cock outlet section 202. Stud 212 extends distally away from the valve body 176.

A metal cap 214 threaded over stud 212 holds the valve cock 188 in bore 178. Cap 214 has a proximal-facing sleeve 216 (shown in phantom) that is threaded over stud 212. A rubber grommet 218 is fitted against the distal-facing end of the valve body 176 and surrounds both the valve cock stud 212 and cap sleeve 216. Grommet 218 is formed to have cylindrically shaped collar 220 that is spaced away from the outer surface of cap sleeve 216. A coil spring 222 extends between the distal facing surface of grommet 218 and the inner surface of cap 214. Spring 222 is located around the outside of cap sleeve 216 and is partially disposed within grommet collar 220. Spring 222 urges the cap 214, and thus the valve cock 188, in the distal direction. Thus, the spring 222 urges the valve cock outlet section 202 against the inner wall of the tapered inner wall of the valve body 176 that defines the distal end of bore 178.

A small lever 224 is screw secured to the proximal-facing end of the valve cock 188, (screws not illustrated). Lever 224 provides personnel with the ability to control the rotational position of the valve cock 188 so that fluid flow in and out of the cannula 20 can be regulated.

In a surgical procedure, the outer tube 44 of the cannula 20 of this invention is fitted in a portal formed in a patient with a trocar according to conventional surgical procedure. Then, the inner tube 46 is fitted in the outer tube 44. More specifically, the inner tube 46 is slid into the outer tube 44 until the inflow plug nose 147 seats in the complementary shaped void space 82 formed in the outflow cap 72. This seating arrangement prevents the inner tube 46 and components attached to it from rotating relative to the outer tube 44. During the insertion of the inner tube 46 in the outer tube 44, the outflow slide 86 initially snaps over the distal end of the inflow plug body 126. Once the inner tube 46 is fully seated in the outer tube 44, O-ring 98 forces the inner edge of slide plate 90 that defines opening 92 into a plug body groove 148. The seating of slide 86 in groove 148 releasably secures the inner tube 46 and components attached to it to the outer tube 44.

Due to its non-circular profile, when the inner tube 46 is seated in the outer tube, the opposed outer surfaces of the inner tube 46, abut the adjacent inner surface of outer tube 44 as illustrated in FIG. 16. Thus, the inner tube 46 is self centered and stabilized within the outer tube 44. As seen in FIG. 16 though, the inner tube 46 does not abut the adjacent inner wall of the outer tube 44 around the complete circumferences of the tubes. Instead, two spaced channels 226 are defined by the interstitial space between the tubes 44 and 46. Channels 226 extend the length of the outer tube and open into the annular space 129 in inflow plug bore 128 that surrounds inner tube sections 102 and 104. Collectively channels 226 and space 129 form the initial parts of the outflow fluid path through the cannula 20.

The endoscope 24 is inserted in the inner tube 46 through outer cap 152. Slide lock 150 holds the endoscope 24 in the cannula 20. The outer diameter of the endoscope 24 is less than the diameter of the bore of inner tube 46. Thus, there is a space in the inner tube 46 that surrounds the endoscope 24. This annular space is the conduit through which irrigation fluid is introduced into the surgical site through the cannula 20.

Valve assembly 50 controls both the fluid flow from the inlet luer fitting 52 and the flow out from the cannula 20 through luer fitting 54. Normally, it is anticipated, valve cock 188 will be set to a full irrigation/no suction position. When the valve cock is so positioned, there is unrestricted irrigation flow from inflow line 30 through the valve assembly into the cannula 20 and to the surgical site. Specifically, the irrigation fluid flows through the valve body 176, valve cock bore 198, inflow housing apertures 122 and the inner tube 46 to the surgical site. When the valve cock is in this state, outlet section 202 blocks fluid flow through outlet hole 182.

During an endoscopic surgical procedure, the suction is drawn through the powered handpiece 36. This prevents excessive fluid build up at the surgical site.

There are times when blood and other debris may cloud the surgical site. At these times, additional suction is required to flush out the fluids at the site. This flushing is accomplished by actuating the lever 224 so as to cause the rotation of the valve cock 188. Bore 204 is shaped so that, as the valve cock 188 is rotated away from the full irrigation/no suction position, the flow through the bore 204 increases almost linearly until the valve cock 188 is in the opposed position, a no irrigation/full suction position.

The rotation of the valve cock from the full irrigation/no suction position towards the no irrigation/full suction position does not, at least initially, result in the reduction of the flow of irrigation fluid through the cannula 20 to the surgical site. Owing to flared profile of the ends of valve cock bore 198, the initial rotation of the valve cock 188 does not result in the blocking of the irrigation fluid flow through the valve body 176. More particularly, owing to the geometry of the valve bore 198, full irrigation flow is maintained through the valve assembly as the valve cock is rotated until it is approximately 65° from the full irrigation/no suction position. Rotation of the valve cock from this intermediate position results in the rapid closing of the irrigation inflow path as the valve cock approaches the no irrigation/full suction position. The complete range of rotation of the valve cock 188 between its full irrigation/no suction and no irrigation/full suction positions is 90°.

The cannula 20 of this invention serves three different functions; it is a guide for the insertion of the endoscope 24 or other instrument into the patient; it defines a flow path through which irrigation fluid can be introduced into the surgical site; and it defines a flow path through which materials can be drawn from the surgical site. Thus, an advantage of this cannula is that it eliminates the need to form plural portals in the patient for receiving separate cannulae that, collectively, perform the above functions.

The cannula 20 of this invention also has a single valve assembly 50 for controlling fluid flow into and out of the surgical site through the cannula. Thus, the surgeon only has to actuate a single device, lever 224, to regulate these flows. Thus, the surgeon does not have to divert his/her concentration to remember which control member must be actuated in order to accomplish the desired fluid control. Furthermore, owing the geometry of the valve cock 188, full irrigation flow is maintained while the surgeon is able to significantly increase the suction flow out of the surgical site. Thus, during the procedure in which suction is increased to clear the view at the surgical site, large amounts of fluid are not drawn away from the site. Thus, during the clearing operation, significant volumes of fluid remain at the site so as to hold the site at the desired pressure. Consequently, when manipulating the valve assembly 50 of this invention the surgeon does not have to devote a considerable fraction of his/her attention and time to setting the valve in order to accomplish the desired flushing of fluid from the surgical site.

It should be realized that the foregoing description is limited to one specific embodiment of the invention. It will be apparent, however, from the description of the invention that the invention can be practiced using alternative components other than what has been described. For example, the cannula of this invention need not always be used as a guide for an endoscope 24. In some versions of the invention, the cannula may not be used as the guide for other instruments that are directed towards the surgical site. Furthermore, in some versions of the invention, the cannula may not even be dimensioned to serve as a guide for surgical instruments. Thus, the cannula is provided with two tubes, each of which functions as a separate one of the inflow and outflow conduits. In these versions of the invention, the above described single valve assembly is employed to regulate fluid flow through two tubes that form the cannula. These tubes may be parallel or concentric.

In still other versions of the invention, a supplemental tube that is parallel or concentric with the inner and outer tubes 44 and 46, respectively, may be provided. This tube may function as a conduit that leads to a pressure sensor. The pressure sensor is employed to provide an indication of fluid pressure at the surgical site. Data representative of this pressure is feed back to the fluid management system 28. Based on this data, the fluid management system regulates the pumping of irrigation fluid to and suction of fluid from the surgical site.

Also, in other versions of the invention, the geometry of the bores formed in valve cock 188 may be different from what has been described. In some versions of the invention, it may not be necessary to shape the valve bore through which the irrigation fluid is flowed into the patient so that the valve is fully open for a substantial portion of the displacement of the valve cock. Moreover, in other versions of the invention, the angular range of motion and open/closed states of the valve may be different from what has been described. It is anticipated that in other versions of the invention, the full range of motion of the valve may vary from 60 to 180°.

Moreover, alternative constructions of the valve may be provided. For example, in one version of the invention, the valve assembly may consist of a valve body that surrounds the inner and outer tubes. Inside the valve body there is a static, sleeve-shaped valve frame that also surrounds the proximal ends of the tubes. A ring shaped valve member surrounds either the inner or outer wall of the valve frame. Both the valve frame and valve member are formed with openings. The extent to which the rotation of the valve member places its openings in or out of registration with the valve frame openings regulates the extent to which the irrigation and suction flows through the tubes are allowed to flow unimpeded or blocked. In this version of the invention, the valve body essentially functions as the collet.

Also, in some versions of the invention, the valve cock may not be rotatably fitted in the associated valve body. For example, in some versions of the invention, the valve cock may be configured to slide in order to achieve the desired fluid flow paths.

Similarly, the valve may be constructed so that irrigation flow does not start to be reduced from its full flow state until the valve has transited between 40 to 90 percent of its full range of motion.

Moreover, in the described version of the invention, collet 48 is rotatable around the outer and inner tubes 44 and 46, respectively. This feature of the invention allows the surgeon to move the collet so that the distal ends of the inflow and outflow lines 30 and 34, respectively, can be placed in position in which they are least obtrusive for the procedure being performed. In other versions of the invention, flexible, shape holding metal tubing may function as the inlet and outlet connections for the valve assembly. In these versions of the invention, the surgeon sets the position of these tubes so they are positioned as unobtrusively as possible. Therefore, in these versions of the invention, it may not be necessary to mount the valve assembly so it is rotatable relative to the inner and outer tubes.

Furthermore, in other versions of the invention, inner tube 46 may have a geometry different from what has been described. For example, in some versions of the invention, the distal portion of the inner tube may simply have a circular cross sectional profile. In these versions of the invention either fins mounted to the outside of the inner tube or dimples formed in the outer tube 44 may be employed to center the tubes 44 and 46 relative to each other. Also, the inner tube may be the member formed with longitudinally spaced-apart dimples that center the inner tube 46 in the outer tube 44.

Also, in some versions of the invention, the moving member that releasably holds the inner tube 46 and valve assembly 50 the outer tube 44 may engage a portion of the inner tube. Alternatively, this moving member may be attached to the inner tube 44, the inflow head 110 or the inflow housing 44 and releasably engage the outer tube 46 or a member attached to the outer tube.

It should likewise be recognized that other fastening means for releasably holding the inner tube in the outer tube and an endoscope in the inner tube may be employed. These assemblies, could, for example, include snap lock mechanisms. Mechanisms that have biased ball bearings and/or mechanisms that facilitated the threaded securement of these components to each other.

Therefore, it is the object of the appended claims to cover all such modifications and variations as come within the true spirit and scope of the invention.

1-27. (Cancelled)

28. A cannula, said cannula comprising:
a first tube having opposed open proximal and distal ends;
a second tube, said second tube having: a distal section that is disposed in said first tube, the distal section having an open distal end, the first tube and the distal section of said second tube being collectively dimensioned to define a first fluid conduit between said tubes; and a proximal section integral with said distal section that is located outside of and extends away from the proximal end of said first tube, the proximal section having an open proximal end wherein, said second tube defines a second fluid conduit that extends through said second tube;
a first locking member moveably attached to one of said first tube or said second tube for engaging and releasably holding said second tube in said first tube; and
a valve assembly including:
a valve body attached to the proximal section of said second tube, said valve body having: a first through hole that extends to and is in fluid communication with the first fluid conduit; and a second through hole that extends to and is in fluid communication with the open proximal end of said second tube; and
a single valve member moveably mounted in said valve body so as to intersect the first through hole and the second through hole, said valve member having a plurality of bores positioned to selectively go in and out of registration with the first and second through holes so that movement of said valve member results in simultaneous regulation of fluid flow through both the first through hole and the second through hole.

29. The cannula of claim 28, wherein said valve body is rotatably mounted to said second tube.

30. The cannula of claim 28, wherein at least a portion of said second tube distal section is shaped to have a non-circular profile and said first and second tubes are dimensioned so that spaced-apart portions of said second tube distal section abut an inner wall of said first tube.

31. The cannula of claim 28, wherein one of said bores is a first bore that is selectively placed in registration with the first through hole of said valve body and another of said bores is a second bore that is selectively placed in registration with the second through hole of said valve body, and said valve member bores are oriented so that said valve member has a first position in which the first bore is in full registration with the first through hole and the second bore is completely out of registration with the second through hole so that said valve member blocks all fluid flow through the second through hole, and a second position in which the first bore is completely out of registration with the first through hole so that said valve member blocks all fluid flow through the first through hole and the second bore is in full registration with the second through hole.

32. The cannula of claim 31, wherein, the bores of said valve member are shaped so that, as the valve member is partially moved between the first and second positions:
one of the bores of said valve member remains in full registration with the valve body through hole with which the bore is associated; and
the remaining one of the bores of said valve member moves partially in/out of registration with the valve body through hole with which the bore is associated.

33. The cannula of claim 28, wherein:
a head is disposed over the proximal end of said first tube and the proximal section of said second tube, said head having at least one first aperture that is positioned to be in fluid communication with the first fluid conduit and at least one second aperture that is positioned to be in fluid communication with the open proximal end of said second tube; and
said valve body is mounted to said head so that the first through hole of said valve body is in fluid communication with the at least one first aperture of said head and the second through hole of said valve body is in fluid communication with the at least one second aperture of said head.

34. The cannula of claim 33, wherein said valve body is rotatably mounted to said head.

35. The cannula of claim 33, wherein said head is formed from first and second sections that are separate pieces and that are integrally attached together; and a portion of said second tube that defines the proximal end of said second tube is secured in said head first section.

36. The cannula of claim 33, wherein said first locking member is moveably attached to said first tube and is positioned to releasably engage said head.

37. The cannula of claim 33, wherein:
an outflow body is mounted to the proximal end of said first tube, said outflow body having a bore in which said tube is mounted and a counterbore concentric with the bore; and
said head has a distally-directed nose that is seated in the counterbore of said outflow body.

38. The cannula of claim 28, wherein:
a head is attached to said second tube and is disposed over the proximal end of said first tube and the proximal section of said second tube, said head having at least one first aperture that is positioned to be in fluid communication with the first fluid conduit and at least one second aperture that is positioned to be in fluid communication with the open proximal end of said second tube;
a collet is rotatably mounted to said head, said collet being formed to have a first bore that is in fluid communication with the at least one first aperture of said head and a second bore that is in fluid communication with the at least one second aperture of said head; and
said valve body is mounted to said collet so that the first through hole of said valve body is connected to the first bore of said collet and the second through hole of said valve body is connected to the second bore of said collet.

39. The cannula of claim 28, further including a second locking member moveably attached to said second tube for releasably holding an instrument in said second tube.

40. A cannula, said cannula comprising:
a first tube having opposed proximal and distal ends, the distal end being open, said first tube defining a first conduit;
a second tube adjacent said first tube, said second tube having a proximal end that is open and adjacent the proximal end of said first tube and a distal end opposite the proximal end that is open and adjacent the distal end of said first tube, said second tube defining a second conduit; and
a valve assembly, said valve assembly having:
a valve body that is attached to at least the proximal end of said second tube and that is shaped to have a first flow hole that extends to the first conduit and a second flow hole that extends to the second conduit; and
a single valve member moveably disposed in said valve body and positioned to intersect the first flow hole and the second flow hole, said valve member being formed with a first bore positioned to selectively be in registration with the first flow hole and a second bore positioned to selectively be in registration with the second flow hole so that said valve member simultaneously controls fluid flow through the first and second flow holes.

41. The cannula of claim 40, wherein:
said second tube has a distal section that is disposed inside said first tube and the distal section of said second tube has an outer diameter less than an inner diameter of said first tube so as to define an interstitial space between said first and second tubes, the interstitial space being the first conduit, and said second tube has a proximal section located outside of the proximal end of said first tube; and
a first locking member is moveably attached to one of said tubes for releasably holding said second tube in said first tube.

42. The cannula of claim 41, further including a second locking member moveably attached to the proximal end of said second tube, said second moveable locking member having a locking element positioned to releasably hold an instrument in said second tube.

43. The cannula of claim 41, wherein said second tube is shaped so that a section of second tube disposed inside said first tube has a non-circular profile and said tubes are dimensioned so that spaced apart portions of said second tube abut an inner wall of said first tube.

44. The cannula of claim 41, wherein:
a head is attached to said proximal section of said second tube, said head having a sleeve that is disposed over and spaced away from an outer wall of said second tube so as to define between said second tube and said sleeve a third conduit that is in fluid communication with said first conduit and said sleeve is formed with at least one aperture that extends to third conduit; and
said valve body is connected to said head so that the first flow hole is in fluid communication with the at least one aperture of said sleeve.

45. The cannula of claim 41, wherein:
a head assembly is attached to the proximal section of said second tube, said head assembly having:
a first sleeve that extends over a portion of said second tube that extends out of said first tube, said first sleeve and said second tube being dimensioned to define therebetween a third conduit that is in fluid communication with said first conduit and said first sleeve is shaped to have at least one first aperture that opens into to said third conduit; and
a second sleeve that is attached to said first sleeve and that extends forward from the proximal end of said second tube and said second sleeve is formed with at least one second aperture that opens into the second conduit; and
said valve body is attached to said head assembly so that the first flow hole extends to the at least one first aperture and the second flow hole extends to the at least one second aperture.

46. The cannula of claim 40, wherein said valve member bores are oriented so that said valve member has a first position in which the first bore is in full registration with the first flow hole and the second bore is completely out of registration with the second flow hole so that said valve member blocks all fluid flow through the second flow hole, and a second position in which the first bore is completely out of registration with the first flow hole so that said valve member blocks all fluid flow through the first flow hole and the second bore is in full registration with the second flow hole.

47. The cannula of claim 46, wherein, the bores of said valve member are shaped so that, as the valve member is partially moved between the first and second positions:
one of the bores of said valve member remains in full registration with the valve body flow hole with which the bore is associated; and
the remaining one of the bores of said valve member moves partially in/out of registration with the valve body flow hole with which the bore is associated.

48. The cannula of claim 40, further including an instrument locking member moveably attached to the proximal end of one of said tubes for removably holding an instrument in said tube.

49. A cannula, said cannula comprising:
an inner tube having a proximal end and a distal end spaced from and opposite the proximal end;
an elongated head assembly disposed over the proximal end of said inner tube, said head assembly having:
a proximal section that extends beyond the proximal end of said inner tube and at least one first aperture that opens into the proximal end of said inner tube;
a sleeve-shaped distal section that extends from the proximal section over a portion of said inner tube adjacent the proximal end of said inner tube, said inner tube and said head assembly distal section being shaped so as to be spaced apart from each other so as to define a first channel between said inner tube and said head assembly distal section and said head assembly distal section is further shaped to define at least one second aperture that extends to the first channel; and
a valve assembly mounted to said head assembly, said valve assembly having: a valve body that is connected to said head assembly and a single valve member that is moveable relative to said valve body and configured to simultaneously regulate fluid flow through the at least one first aperture and the at least one second aperture of said head assembly; and
an outer tube attached to said head assembly distal section and that extends over a portion of said inner tube that extends distally from said head section, said inner tube and said outer tube being dimensioned so as to be spaced apart from each other so as to define a second channel that is contiguous with the first channel.

50. The cannula of claim 49, wherein:
a collet is disposed over said head assembly, said collet having a first port that extends to the at least one first aperture of said head assembly and a second port that extends to the at least one second aperture of said head assembly; and
said valve body is mounted to said collet and said valve member regulates fluid flow through the first port and the second port of the collet.

51. The cannula of claim 50, wherein said collet is rotatably mounted to said head assembly.

52. The cannula of claim 49, wherein said valve body is rotatably mounted to said head assembly.

53. The cannula of claim 49, further including a tube locking member attached to said head assembly or to said outer tube for releasably holding said inner tube in said outer tube.

54. The cannula of claim 49, wherein: said outer tube has a proximal end that is located adjacent the head assembly distal section; and a tube locking member is moveably attached to the proximal end of said outer tube and is positioned to releasably engage a portion of said inner tube or said head assembly.

55. The cannula of claim 49, wherein a portion of said inner tube disposed in said outer tube is shaped to have a non-circular profile and said inner tube and said outer tubes are dimensioned so that spaced-apart portions of said inner tube abut an inner wall of said outer tube.

56. The cannula of claim 49, wherein:
said valve body is formed to have a first through hole that extends to the at least one first aperture of said head assembly and a second through hole that extends to the at least one second aperture of said head assembly; and
said valve member is moveably fitted in said valve body member and is positioned to intersect the first and second through holes of said valve body and said valve member is formed to have a first bore that is selectively placed in registration with the first through hole of said valve member and a second bore that is selectively placed in registration with the second through hole of said valve member, and said valve member bores are oriented so that said valve member has a first position in which the first bore is in full registration with the first through hole and the second bore is completely out of registration with the second through hole so that said valve member blocks all fluid flow through the second through hole, and a second position in which the first bore is completely out of registration with the first through hole so that said valve member blocks all fluid flow through the first through hole and the second bore is in full registration with the second through hole.

57. The cannula of claim 56, wherein, the bores of said valve member are shaped so that, as the valve member is partially moved between the first and second positions:
one of the bores of said valve member remains in full registration with the valve body through hole with which the bore is associated; and
the remaining one of the bores of said valve member moves partially in/out of registration with the valve body through hole with which the bore is associated.

58. The cannula of claim 49, further including an instrument locking member moveably attached to the proximal section of said head assembly for releasably holding an instrument in said inner tube.

59. A surgical cannula arrangement comprising:
a first tube having spaced proximal and distal ends and defining a first fluid conduit therein;
a second tube disposed within said first tube and having spaced proximal and distal ends, said second tube defining a second fluid conduit therein; and
a valve assembly including a valve body positioned at said proximal ends of said first and second tubes and defining a first port in communication with said first fluid conduit and a second port in communication with said second fluid conduit, and a valve member movably disposed within said valve body, said valve member defining therein first and second bores respectively positioned on said valve member for selective communication with said first and second ports, said valve member having a first position relative to said valve body wherein said first bore is in communication with said first port to allow fluid flow through said first fluid conduit and said second bore is positioned in non-communication with said second port to prevent fluid flow through said second fluid conduit, said valve member having a second position relative to said valve body wherein said second bore is in communication with said second port to allow fluid flow through said second fluid conduit and said first bore is positioned in non-communication with said first port to prevent fluid flow through said first fluid conduit, and said valve member having an intermediate position between said first and second positions wherein said first and second bores are both in communication with the respective first and second ports to allow fluid flow through said first and second conduits simultaneously.

60. The surgical cannula of claim 59, wherein said first fluid conduit is defined between an outer surface of said second tube and an inner surface of said outer tube, said first fluid conduit defining a suction conduit for suctioning fluid from a surgical site, and said second fluid conduit defining an irrigation conduit for supplying irrigation fluid to the surgical site.

61. The surgical cannula of claim 59, wherein said valve member defines a longitudinal axis, and said first and second bores of said valve member are spaced from one another along the longitudinal axis.

62. The surgical cannula of claim 59, wherein each said first and second bore of said valve member defines a bore axis, said bore axes of the respective first and second bores being transversely oriented relative to one another.

63. The surgical cannula of claim 59, wherein said valve member is generally cylindrical in shape and defines an outer peripheral surface, each said first and second bore of said valve member opening outwardly through said outer peripheral surface at opposite ends thereof, said ends of one of said first and second bores defining respective circumferential flares at said outer peripheral surface.

64. The surgical cannula of claim 63, wherein said circumferential flares of said one bore are disposed to partially circumferentially overlap the respective ends of the other said bore to allow fluid flow through said first and second conduits in said intermediate position of said valve member.

65. The surgical cannula of claim 59, wherein one of said first and second bores of said valve member is flared at opposite ends thereof as said ends open through an outer peripheral and generally cylindrical surface of said valve member, and the other said bore is cylindrical in shape.

66. The surgical cannula of claim 59, wherein said valve member is rotatable relative to said valve body through an approximately ninety-degree angle between said first and said second positions, and wherein said valve member allows both maximum flow of fluid through said second fluid conduit when in said second position and maximum flow of fluid through said second fluid conduit through a substantial portion of its travel from said second position to said first position, and said valve member allows a substantially linear increase in the flow of fluid in said first fluid conduit as said valve member is moved from said second position towards said first position.

67. The surgical cannula of claim 66, wherein said first fluid conduit is defined between said inner and outer tubes and defines a suction conduit, and said second fluid conduit defines an irrigation conduit.

68. A surgical cannula arrangement comprising:
a first tube having spaced proximal and distal ends and defining a first fluid conduit therein;
a second tube having spaced proximal and distal ends and defining a second fluid conduit therein; and
a valve assembly positioned adjacent said proximal ends of said first and second tubes, said valve assembly including a valve body defining therein a first port in communication with said first fluid conduit and a second port in communication with said second fluid conduit, and a valve member disposed within said valve body for movement relative thereto, said valve body defining an outer peripheral surface, a first bore positionable in communication with said first port to allow fluid flow within said first fluid conduit and a second bore positionable in communication with said second port to allow fluid flow through said second fluid conduit, said first and second bores opening through said outer peripheral surface of said valve body in partial circumferential overlapping relation with one another to permit simultaneous fluid flow through both said first and second fluid conduits.

69. The surgical cannula of claim 68, wherein said first tube defines a longitudinal axis and said second tube is disposed coaxially within said first tube, said first fluid conduit being defined between said first and second tubes.

70. The surgical cannula of claim 69, wherein each said first and second bore of said valve member has a pair of opposite ends which open through said outer generally cylindrical peripheral surface of said valve member, said opposite ends of one of said first and second bores being circumferentially flared to provide said partial circumferential overlapping relation.

71. The surgical cannula of claim 68, wherein said first and second bores are spaced longitudinally from one another along said valve member and define respective bore axes which are oriented transversely relative to one another.

72. The surgical cannula of claim 68, wherein said valve member is rotatably movable within and relative to said valve body between a first limit position wherein said first bore is in communication with said first port to allow fluid flow within said first fluid conduit and said second bore is blocked from communication with said second port, and a second limit position wherein said first bore is blocked from communication with said first port and said second bore is in communication with said second port to allow fluid flow through said second fluid conduit.

73. The surgical cannula of claim 72, wherein said valve member has a position between said first and second positions wherein said first and second bores of said valve member are both in communication with the respective first and second ports to permit said simultaneous fluid flow.

74. A surgical cannula arrangement comprising:
an elongated outer tube having spaced proximal and distal ends and defining a longitudinal axis;
an elongated inner tube having spaced proximal and distal ends, said inner tube being disposed coaxially within said outer tube and therewith defining a first fluid conduit which extends between an outer surface of said inner tube and an inner surface of said outer tube, said inner tube further defining a second fluid conduit therein which is separated from said first fluid conduit by said inner tube; and
a valve assembly disposed at said proximal ends of said inner and outer tubes, said valve assembly including a valve body defining therein axially-spaced first and second ports which respectively communicate with said first and second fluid conduits, and a valve member disposed within said valve body for rotation relative thereto, said valve member defining therein first and second bores which are axially aligned with said first and second ports, said valve member being rotatable into a first position relative to said valve body wherein said first bore is in full communication with said first port to allow maximum fluid flow through said first fluid conduit and said second bore is out of communication with said second port to prevent fluid flow through said second fluid conduit, a second position relative to said valve body wherein said second bore is in full communication with said second port to allow maximum fluid flow though said second fluid conduit and said first bore is out of communication with said first port to prevent fluid flow through said first fluid conduit, and third position relative to said valve body wherein said first and second bores are both in communication with the respective first and second ports.

75. The surgical cannula of claim 74, wherein said first fluid conduit is a suction conduit for providing suction at a surgical site, and said second fluid conduit is an irrigation conduit for providing irrigation fluid to a surgical site.

76. The surgical cannula of claim 74, wherein each said first and second bore of said valve member opens at opposite ends thereof through an outer generally cylindrical surface of said valve member, said ends of one of said first and second bores being flared to provide said one bore with a non-cylindrical shape and the other said bore being cylindrical in shape.

77. The surgical cannula of claim 74, wherein said first and second bores open through an outer peripheral surface of said valve member in circumferential overlapping relation with one another, and axes of the respective said first and second bores are oriented transversely.

78. The surgical cannula of claim 74, wherein one of said first and second bore of said valve member is circumferentially elongate as same opens through an outer peripheral surface of said valve member.

79. A surgical cannula comprising:
a first tube having spaced proximal and distal ends and defining a first fluid conduit therein;
a second tube disposed within said first tube and having spaced proximal and distal ends, said second tube defining a second fluid conduit therein; and
a valve assembly including a valve body positioned at said proximal ends of said first and second tubes and defining a first port in communication with said first fluid conduit and a second port in communication with said second fluid conduit, and a valve member movably disposed within said valve body and defining therein first and second bores respectively positioned on said valve member for selective communication with said first and second ports, said valve member being movable relative to said valve body into a position wherein said first and second bores are respectively positioned in communication with said first and second ports to permit fluid flow within both said first and second fluid conduits simultaneously.

80. The surgical cannula of claim 79, wherein said first fluid conduit is defined between an outer surface of said second tube and an inner surface of said first tube and defines a suction conduit, and said second fluid conduit defines an irrigation conduit.

81. The surgical cannula of claim 79, wherein said valve member is rotatably movable relative to and within said valve body between a first limit position wherein said first bore is in communication with said first port and said second bore is blocked from communication with said second port, and a second limit position wherein said first bore is blocked from communication with said first port and said second bore is in communication with said second port, and said position of said valve member is disposed between said first and second limit positions.

82. The surgical cannula of claim 79, wherein said valve member defines a longitudinal axis and said first and second bores are longitudinally spaced from one another along said valve member, each said first and second bore defining a bore axis, said bore axes being oriented transversely to one another.
